(19) United States
(12) Patent Application Publication
(10) Pub. No.: US 2023/0000269 A1
(43) Pub. Date: Jan. 5, 2023

(54) PILLOW WITH PROXIMAL AIR FLOW DELIVERY MANIFOLD

(71) Applicant: James A. Waring, Libertyville, IL (US)

(72) Inventor: James A. Waring, Libertyville, IL (US)

(21) Appl. No.: 17/857,617

(22) Filed: Jul. 5, 2022

Related U.S. Application Data
(60) Provisional application No. 63/218,397, filed on Jul. 4, 2021.

(51) Int. Cl. A47G 9/10; A61M 16/00

(52) U.S. Cl. CPC A47G 9/10; A61M 16/0003; A61M 2202/0208

(57) ABSTRACT

Aspects of the invention include devices, systems, and methods for enhancing a user's sleep. A flexible frame supports a flow manifold which can be used in unison with adjoined or adjacently integrated supportive members utilized to create soft objects of various designs and materials. These types of objects can be in the form of a pillow, mattress topper, even a child's stuff animal and other various stationary and portable soft sided padded embodiments as disclosed in this application. These various designs and embodiments house an integrated flow delivery system which is delivers concentrated, pressurized, and or altered gaseous flowing elements in a controlled and fluidic manner as to help address certain types of sleeping and breathing issues during their resting and sleep states.

### CROSS REFERENCE TO RELATED APPLICATION

This application claims benefit of U.S. Provisional Application No. 63/218,397 filed on Jul. 4, 2021 which is incorporated by reference in its entirety.

### TECHNICAL FIELD

The present invention relates to resting devices such as pillows, mattresses, and cushions, and or the associated covers of such devices.

### BACKGROUND

Poor sleep is a global issue. In some countries, around 30% of the adult population suffer from chronic insomnia. In general, people with sleeping disorders are also more likely to have other health issues, some of which may be chronic. People with health issues (e.g., a sleep disorder and another health issue) are more likely to be involved in accidents. For some people, it is challenging to get enough oxygen when sleeping due to breathing patterns changing during the resting state. Getting insufficient oxygen during sleep can result in hypoxia and/or hypoxemia. A variety of sleep aid technologies exist to help a person get sufficient oxygen when sleeping.

Most of these sleeping aid technologies require direct engagement between an operator and a physical device. Direct engagement can add additional issues of comfort and usability layered upon those in which the operator or user wishes to initially solve. Some types of devices penetrate into a user's nostrils and are used to open up nasal passages allowing the user to breath larger amounts of air. However, it is easy for a user to inadvertently remove these type of devices during sleep due, mitigating any benefits the device might have provided.

Other types of devices either keep the mouth open (e.g., mouth guards) or keep the mouth closed (e.g., head wraps) physically altering the mechanical construct of the user's body. However, such physical alterations can cause continued and ongoing discomfort resulting in difficulty while attempting to relax and fall asleep. The instructions on these other types of devices state time is needed for one to get used to using them, which reduces the likelihood of them being an immediate solution even if they were effective.

Further types of devices are strapped onto the operator user's mouth and nose to pressurize the air introduced directly into their body (e.g., a CPAP machine).

Many devices have the potential to introduce pathogens and other harmful matter into inspired aired. CPAP machines often include basic air filters. However, CPAP machines can be uncomfortable and even feel claustrophobic to some wearers. CPAP tubing can also impede movement making sleep difficult. Depending on mask configuration, a mask can create pressure sores and it is generally annoying wearing a mask while sleeping. CPAP machines also introduce a myriad of other well-known potentially harmful issues associated with general machine cleanliness and maintenance issues.

Research into relaxation and general feeling of wellbeing associated with rest has explored other mechanisms, including introducing sounds or smells into a sleeping environment. For example, white noise, soothing rhythmical sounds, and therapeutic aromas have been found to promote restful states. Sleeping environment quality, for example, air temperature, humidity level, etc., can also be controlled to reduce particle counts and unpleasant or harmful Volatile Organic Compounds (VOCs).

As such, people with health issues or that are seeking ways to have their body and mind to recover are forced to choose from a multitude of technologies and or machines when attempting to address factors which effect one's perception of wellbeing.

### SUMMARY

Aspects of the invention include devices, systems, and methods for enhancing a user's sleep. A flexible frame supports a flow manifold which can be used in unison with adjoined or adjacently integrated supportive members utilized to create soft objects of various designs and materials. These types of objects can be in the form of a pillow, mattress topper, even a child's stuff animal and other various stationary and portable soft sided padded embodiments as disclosed in this application. These various designs and embodiments house an integrated flow delivery system which is delivers concentrated, pressurized, and or altered gaseous flowing elements in a controlled and fluidic manner as to help address certain types of sleeping and breathing issues during their resting and sleep states.

### DESCRIPTION OF THE DRAWINGS

In order to describe the manner in which the above-recited and other advantages and features can be obtained, a more particular description will be rendered by reference to specific implementations thereof which are illustrated in the appended drawings. Understanding that these drawings depict only some implementations and are not therefore to be considered to be limiting of its scope, implementations will be described and explained with additional specificity and detail through the use of the accompanying drawings in which:

FIG. 1 is a top view of a pillow.

FIG. 2 is a top view of a pillowcase

FIG. 3 is a partially transparent top view of the pillow

FIG. 4 is a partially transparent top view of the pillow and the pillowcase.

FIG. 5 is a front view of gaseous fluidic transfer device.

FIG. 6 is a top view of the gaseous fluidic transfer device components along with additive components

FIG. 7 is partially transparent front view of the pillow and the pillowcase.

FIG. 8 is another partially transparent front view of the pillow and the pillowcase having an upward curved profile.

FIG. 9 is a further partially transparent front view of the pillow and the pillowcase having an upward curved profile with additional fill material.

FIG. 10 is partially transparent front view of the pillow and pillowcase relative to a user's head.

FIG. 11 is partially transparent front view of the pillow and pillowcase with the user's head in contact with the pillow and pillowcase.

FIG. 12 is another partially transparent front view of the pillow and pillowcase with user's head in contact with the pillow and pillowcase.

FIG. 13 is partially transparent front view of the pillow and pillowcase connected to an oxygen cannister.

FIG. 14 is partially transparent front view of the pillow and pillowcase connected to an oxygen source and relative to bed components.

### DETAILED DESCRIPTION

In the Summary above and in this Detailed Description, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

“Exemplary” is used herein to mean “serving as an example, instance, or illustration.” Any aspect described in this document as “exemplary” is not necessarily to be construed as preferred or advantageous over other aspects.

Throughout the drawings, like reference characters are used to designate like elements. As used herein, the term “coupled” or “coupling” may indicate a connection. The connection may be a direct or an indirect connection between one or more items. Further, the term “set” as used herein may denote one or more of any item(s), so a “set of items” may indicate the presence of only one item or may indicate more items. Thus, the term “set” may be equivalent to “one or more” as used herein.

Aspects of the invention provide a quality resting and sleeping experience by improving air quality in the immediate local environment surrounding an individual when resting or sleeping. In one aspect, an air delivery system is integrated into a pillow or other soft item (e.g., matters topper, stuffed animal, etc.). The air delivery system can deliver concentrated, pressurized, and or altered gaseous flowing elements to a user that is resting on or bear the pillow or other soft item. In doing so, the air delivery system is design to give relief to those having difficulty obtaining the quality sleep needed to rest, recover, and heal from daily physical and mentally taxing activities.

Sleeping restfully is problematic to millions of individuals and typically in many cultures, a bed, mattress, or cushion, and pillows are used to support the individual during their sleep and resting state. Due to the commonality of this arrangement and the significant time a person spends attempting to sleep within a set environment, it can be appropriate to utilize these common elements to deliver a healthy and relaxing experience to facilitate one's ability to enter into and maintain a healthy sleeping habit.

Previously disclosed devices and products out on the market deliver treated air directly to an individual's nose and mouth by way of mask and harness or are passive in nature as to be inserted into the mouth or nasal openings in an attempt to keep open the individual's air passageways during sleep. Both of these types of configurations are uncomfortable and have the tendency to impede the relaxed state needed in such a way that it takes a noted amount of time to adjust to these types of devices to benefit from them. In the alternative, other delivery methods are ambiguous and generalized since they are designed to treat the entire house or room versus the individual in a customized and prescriptive manner.

Accordingly, aspects of the invention uniquely address an individual's needs by addressing the immediate proximal space within the targeted individual's environment in such a way as to customize, transform and treat the user's immediately proximal airspace as to create a superior and improved resting and sleeping experience.

One component of the invention includes a flexible or soft supportive framing of which is resiliently collapsible in a manner as to ensure continued and proper fluidic communication and delivery within and or adjacent to the framework while remaining comfortable to the user resting on or adjacent to the flexible supportive frame. The framework itself in one embodiment allows for the fluid transfer within or in an alternative embodiment, adjacent to the frame so the flexible or resiliently collapsible nature of the frame does not impede the efficiency by restricting the fluidic flow within or adjacent to the supportive framing. Such a framework can be the supportive elements such as those composing a pillow and another alternative embodiment is a resilient framework configured to accept a supportive or pillow like structure within its framework thus adaptable to accept a range of supportive forms.

Aspects of the invention adventurously move air directly from the acceleration, pressurization and treating module through a flexible, resilient hollow profile. The resilient hollow profile forms a framing or is adjacent to the framing and by way of an outlet vent assembly proximal to the individual's mouth and nose transfers air without the outlet vent having to make direct contact with the person's mouth or nose. Furthermore, since the fluidic elements travel through an enclosed channel or supportive frame and not the structural material of the pillow, mattress and or cushion itself, allows for an easier to maintain and clean and healthier system.

In its one embodiment, the device has a fluid pressure generator which consists of a powered motor and a fan blade element which by the way of the controller, user interface, changes the motor and fan's on and off state and operational speed of the fan therefore controlling the airspeed and fluidic flow throughout the device. Air drawn into the fan is then circulated into a manifold which can have one, two or a plurality of structural flow tubes which create a semi resilient frame or structure. At the distal end of the flow tubes after the manifold, a vent or outlet is assembled to ensure a controlled directional flow when exiting the vent assembly.

In an alternative embodiment, the intake prior to or post fluid pressure generator, a filter unit such as a HEPA filter is in fluid communication allowing any air moving through the filter to be cleaned of particles, optionally if a carbon material is added, VOCs can be trapped as well. Other filtering methods and materials can be added to capture and transform the original intake once passed through the filtering elements.

Continuing in an alternative embodiment, post filters, an additive stage can be configured whereas an additional element or elements can be added to the fluidly moving air such as formulated gases made to improve one's health or recovery. For example, additional oxygen known to help in recovery from heavy exercise or to reduce snoring can be added by way of a pressurized canister and an adjustable dosing cycle valve in which a controlled or prescribed amount would be added to the stream of air fluidly moving through the system. Other soothing elements such as eucalyptus oil known to provide respiratory relief can be vaporized and added as well. These are only some of the examples of additional additives others, such as vitamin supplements in a vaporized or fluidly aromatic form can be added during this stage.

In another embodiment, the dosing of additional supplemental elements can be regulated by way of the controller, the user setting, an integrated circuit with firmware, and a user input interface in which the user can select the desired settings.

In another embodiment, pressure sensors can be added to ensure the unit only operates when the person is in proximity to the outlet vents. In yet another embodiment, additional sensors can be assembled onto the device as to sense vibrations, sound, and movement to track the user's sleep state profile, store the information and or transmit it by wire or wirelessly to a device such as a phone, tablet, computer or smart watch with a program or application in which the stored information is presented and the device controls could be accessed through the application as well. The information collected would be accessible and presented in a meaningful way to the user by offering user selectable choices. Alternatively, or in unison, information could be stored, accessed, and communicated by way of the apparatus itself using visual, sound, haptic communications, and other means.

Aspects of the invention include a semi rigid and resilient framework which can be interlaced within a supportive element which constructs a pillow, mattress, cushion, or any other soft sided object including a plush object for younger people like toys, animals, and characters. The tube-like assembly has at least one outlet placed in proximity to where the individual would naturally rest their head as to facilitate proximal intake breathing from the device's vented outlet. Alternatively, another embodiment is where the semi rigid hollow assembly structure embedded within an optional padded soft frame which interfaces with most off the shelf standard pillows. Versions which interface with car and seat cushions, mattresses, and or covers, along with tent like structures hereby disclosed as well.

Aspects of in the invention can utilize cooling fluids as well as elements in gas form to create a multitude of embodiments of which have been disclosed. Other aspects, features, and advantages of the invention will become apparent from the disclosed detailed descriptions along with the accompanying drawings. The embodiments described and illustrated herein are exemplary and are not meant to limit the scope of the invention in any way including limiting its portable nature and where the device can be utilized and what form the device takes due to its soft sided frame construction.

FIG. 1 is a top view of pillow 101. In general, pillow 101 can include a gaseous fluidic transfer device assembly 200 (partially visible). The gaseous fluidic transfer device assembly 200 (an air transfer device) intakes and processes gaseous compounds within. The gaseous fluidic transfer device assembly 200 exhausts the transformed elements through and out the unit vents 464 by way of a manifold flow path network. Pillowcase cover 800 has openings 810 to allow vents 464 to protrude through the openings and by way of main optionally zippered, buttoned, hook and loop, controlled opening 820, is configured to be easily taken off for cleaning.

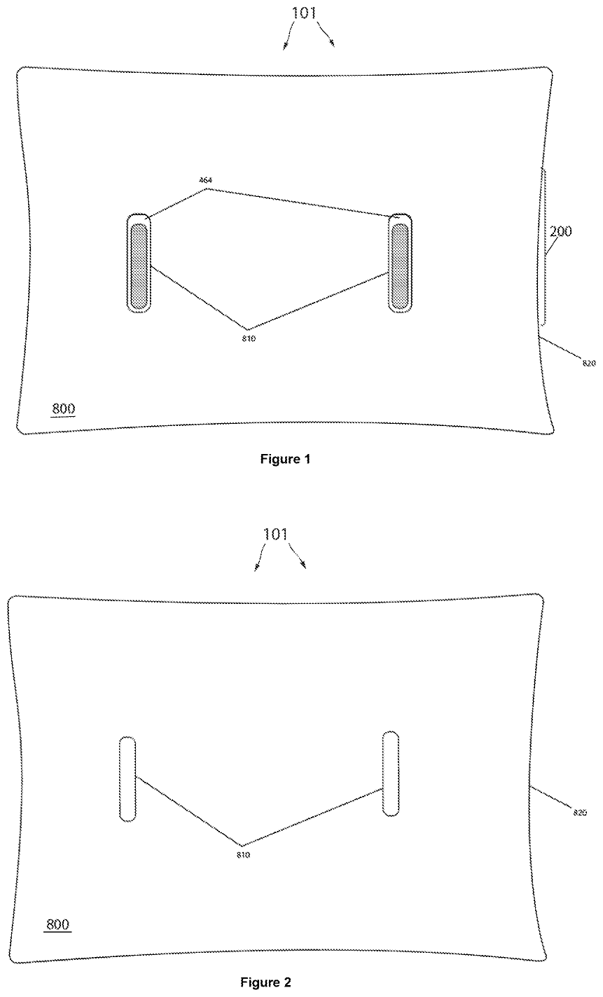

FIG. 2 is a top view of pillowcase 800. Pillowcase 800 can be made of sewn cotton fabric or similar material with openings 810. Openings 810 can be configured with an optional zipper, buttons, hook and loop, or any other similar connectors to control the opening 820.

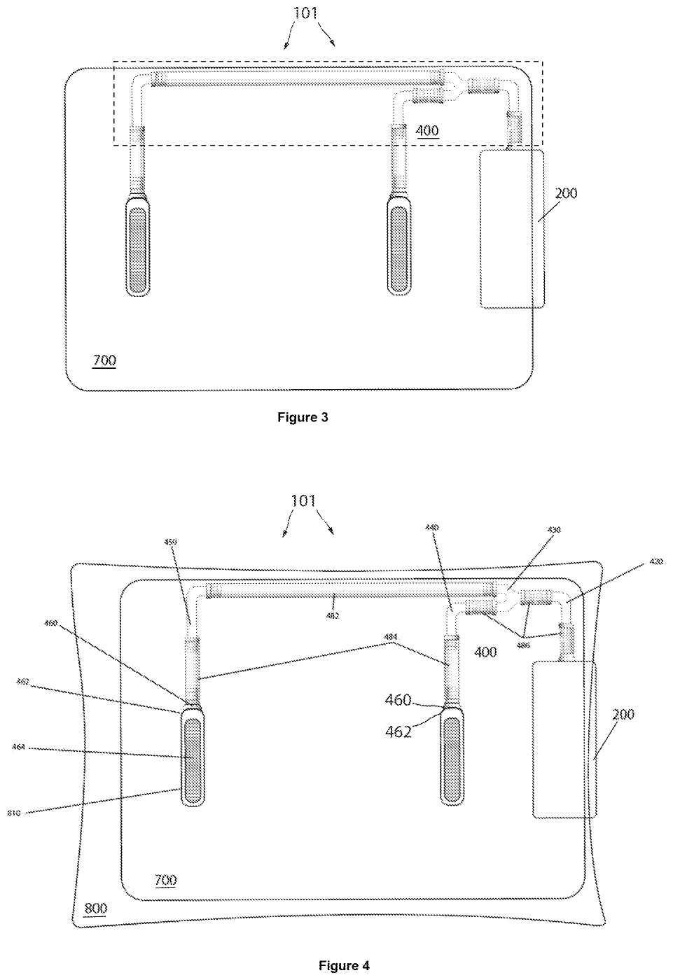

FIG. 3 is a partially transparent top view of pillow 101. As depicted in FIG. 3, pillow 101 and pillowcase 800 are transparent exposing supportive frame 700. Supportive frame 700 is flexible and resilient and can formed from polyurethane foam, polyester fiber type fill materials, or other materials. Alternative materials, such as, organic cotton, recycled polyester or similar materials can be used. The outer shape of supportive frame 700 can be formed or sewn as to contain any loose fill within a desired shape and mass.

Gaseous fluidic transfer device assembly 200 is contained within supportive frame 700. Gaseous fluidic transfer device assembly 200 can intake air. Gaseous fluidic transfer device assembly 200 can alter the air by using a pressurization process. A motorized fan, filtering components and a sub-assembly can be utilized to facilitate the pressurization process. The sub-assembly can accommodate the introduction of additive compounds. The additive compounds can flow through manifold flow path 400 and out unit vents 464 into adjacent open air. Flow through manifold flow path and out vents 464 can be implemented in a controlled manner.

FIG. 4 is a partially transparent top view of the pillow 101 and pillowcase 800. As depicted in FIG. 4, pillow 101 and pillowcase 800 are transparent exposing supportive frame 700. As depicted, manifold flow path assembly 400 includes flexible flow tubes 482, 484, and 486 with connectors 420, 430, 440, and 450. The components of manifold flow path assembly 400 link gaseous fluidic transfer device assembly 200 to pivotal connectors 460. Pivotal connectors 460 can rotate to aid in configuring (e.g., rotating) vents 464 to a desired position towards the user. Vent body 462 has a material retention profile to entrap pillowcase cover opening 810. Vents 464 protrude through openings 810 to bring (e.g., transformed pressurized) air in fluidic communication with the adjacent open environment (e.g., near a user's head).

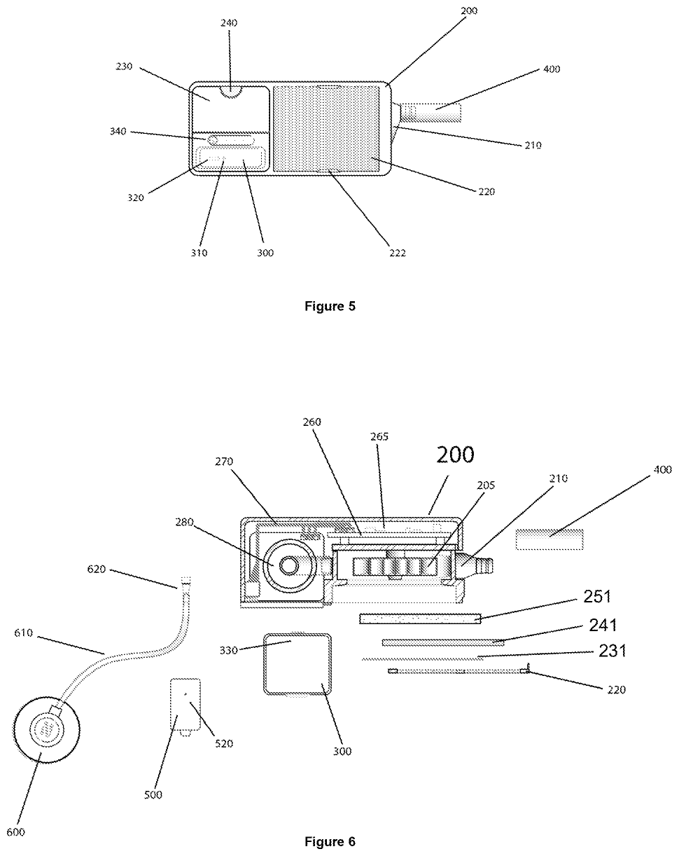

FIG. 5 is a front view of gaseous fluidic transfer device assembly 200. As depicted, gaseous fluidic transfer device 200 incudes unit output 210, intake grill 220, grill retainer 222, access door 230, door actuator 240, battery 300, LED indicator light 310 (e.g., red, yellow, and green), battery charging port 320 (e.g., USB), and control panel 340. Grill retainer 222 helps keep intake grill 220 in place. When actuated, grill retainer 222 allows for grill 220 to be removed providing access to internal air transforming components. Unit output 210 sends transformed fluidic gaseous elements into manifold flow path assembly 400.

Battery 300 can be charged, via battery charging port 320, using a USB plug with wall transformer. The USB plug can directly power gaseous fluidic transfer device assembly 200 or be utilized to charge battery 300. When wall power is not available, battery 300 can power gaseous fluidic transfer device assembly 200.

Control panel 340 can include controls for gaseous fluidic transfer device assembly 200 as well as a user interface. The user interface can present information respecting the condition of gaseous fluidic transfer device assembly 200. The user interface can include colored LEDs (e.g., LED indicator light 310), backlight screens, sounds, graphical interface, etc. Access door 230 includes door actuator 240. Door actuator 240 can be a latch, a finger access detail or of any other similar configuration used to open (and/or close access door 230). When access door 230 is opened, the interior of gaseous fluidic transfer device assembly 200 is accessible.

FIG. 6 is a top view of the gaseous fluidic transfer device components along with additive components. As depicted, gaseous fluidic transfer device assembly 200 includes prefilter 231, carbon filter 241 and HEPA filter 251 behind intake grill 220. Fan and motor assembly 205 can output pressurized accelerated gaseous compounds into exit flow chamber 210 and into manifold flow path assembly 400. PCB assembly 260 and integrated circuit 265 can control fan and motor assembly 205. PCB assembly 260 and integrated circuit 265 can be in electrical communication with fan and motor assembly 205 via wire cable assembly 270. Wire cable assembly 270 can link between PCB assembly 260 and integrated circuit 265 can be in electrical communication with fan and motor assembly 205 via an arrangement of connectors and direct solder joints to control panel 340 and to accessory interface 280. Accessory interface 280 facilitates control of any additional attached accessories and the control processes needed to induce them into the unit's pressurizing and distribution manifold.

For example, accessory interface 280 can be configured to actuate any of a plurality of additional accessories including aroma infuser 500 and oxygen cannister 600. In one aspect, aroma infuser 500 includes actuator 520. Aroma infuser is configured to transform a liquid, such as, for example, eucalyptus, into a gas state. The gas can then be introduced in a controlled manner through interface 280, into fan and motor assembly 205, out unit output 210, and into manifold flow path 400.

In another aspect, oxygen in oxygen canister assembly 600 is transferred through tube 610 and connector 620. The oxygen can then be introduced in a controlled manner through interface 280, into fan and motor assembly 205, out unit output 210, and into manifold flow path 400.

In general, accessory interface 280 can be controlled to induce preprogrammed threshold dose amounts of gases. Accessory interface 280 can be controlled by the user interface, sensed by way of an auto recognition sensor (e.g., a gas sensor (not pictured)), etc. to facilitate appropriate does levels.

Gaseous fluidic transfer device assembly 200 can be powered by a USB plug and wall plug transformer. The USB plug and wall plug transformer can power gaseous fluidic transfer device assembly 200 directly and/or can charge battery 300 with power connecter 330, which in turn powers the gaseous fluidic transfer device assembly 200.

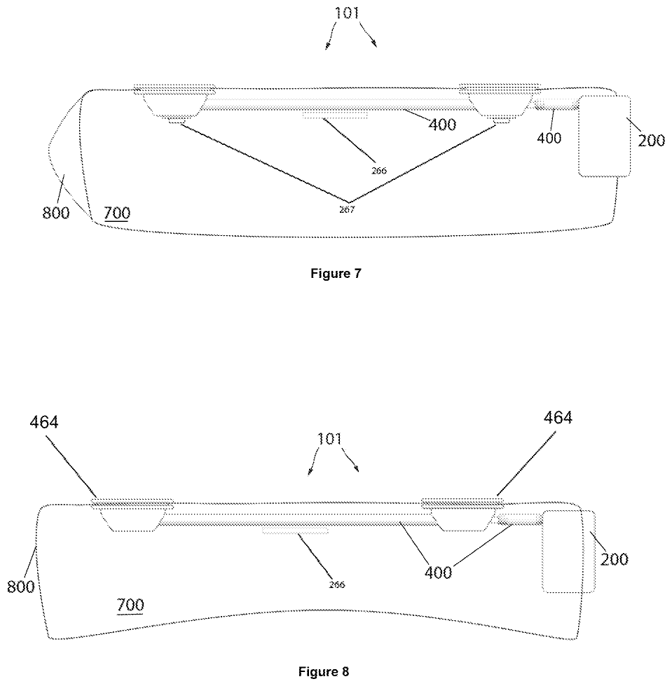

FIG. 7 is partially transparent front view of the pillow 101 and the pillowcase 800. As depicted in FIG. 7, pillow 101 and pillowcase 800 are transparent exposing supportive frame 700. Gaseous fluidic transfer device assembly 200 is depicted in fluidic communication through manifold flow path assembly 400. FIG. 7 further depicts sensor 266 and speakers 267. Sensor 266 can be a presence sensor, pressure sensor, audio sensor or a film contact sensor or any combination mention or of similar functionality. Sensor 266 can detect the displacement of resilient frame 700 and or presence of someone in contact with the frame. Speakers 267 can be in direct, or in wireless communication to a phone, table or any audio communications device. Speaker 267 can be controlled by the audio communications device and/or by components of gaseous fluidic transfer device assembly 200. Speakers 267 can output audio including music, communications and or audio signals to a user to help induce sleep, provide soothing sounds, music, and other signals such as unit state and condition as well as a wakeup sequence.

FIG. 8 is another partially transparent front view of the pillow 101 and the pillowcase 800 having an upward curved profile. As depicted in FIG. 8, pillow 101 and pillowcase 800 are transparent exposing supportive frame 700. In FIG. 8, supportive frame 700 has an upward curved profile on the underside. The upward curved profile can regulate flex. Outlet vents 464 protrude through pillow case opening 810.

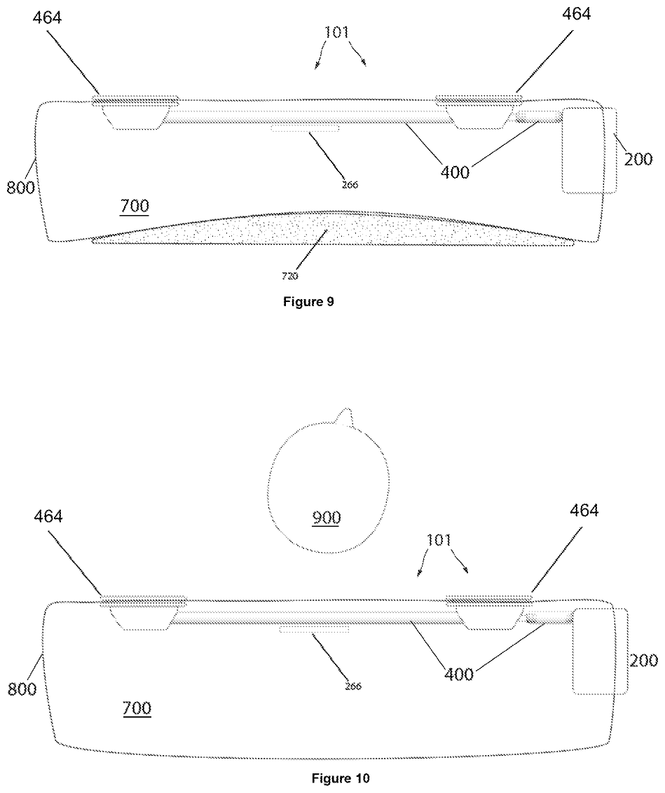

FIG. 9 is a further partially transparent front view of the pillow 101 and the pillowcase 800 having an upward curved profile with additional fill material 720. Inserted into the curved profile, an additional resilient frame piece 720. Resilient frame piece 720 can have differing compression and recovering profile of the material utilized in supportive frame 700. Resilient frame piece 720 can further control the flex and resiliency of pillow 101. Outlet vents 464 protrude through pillow case opening 810.

FIG. 10 is partially transparent front view of the pillow 101 and pillowcase 800 relative to a user's head 900. As depicted in FIG. 10, sensor 266 can be used to detect displacement of supportive frame 700 or presence of something (e.g., head 900) in contact with supportive frame 700. An alternative or supplemental actuator, for instance a tension strap, adjustable and nonadjustable versions can be configured to traverse between both outlet vent assemblies 464 to ensure they adjust to inward positions automatically when the resilient frame 700 is engages with a user. User's head 900 is shown above the device prior to contacting the device.

FIG. 11 is partially transparent front view of the pillow 101 and pillowcase 800 with the user's head 900 in contact with the pillow 101 and pillowcase 800. As depicted in FIG. 11, sensor 266 has detected the displacement of supportive frame 700 and or presence of user's head 900 in contact with supportive frame 700. In response, outlet vent assemblies 464 are rotated inward. Thus, air flow can be focused in a flow pattern surrounding head 900. In general, when supportive frame 700 is displaced or compressed downward from a user's body weight, directional flow can be enhanced towards the user.

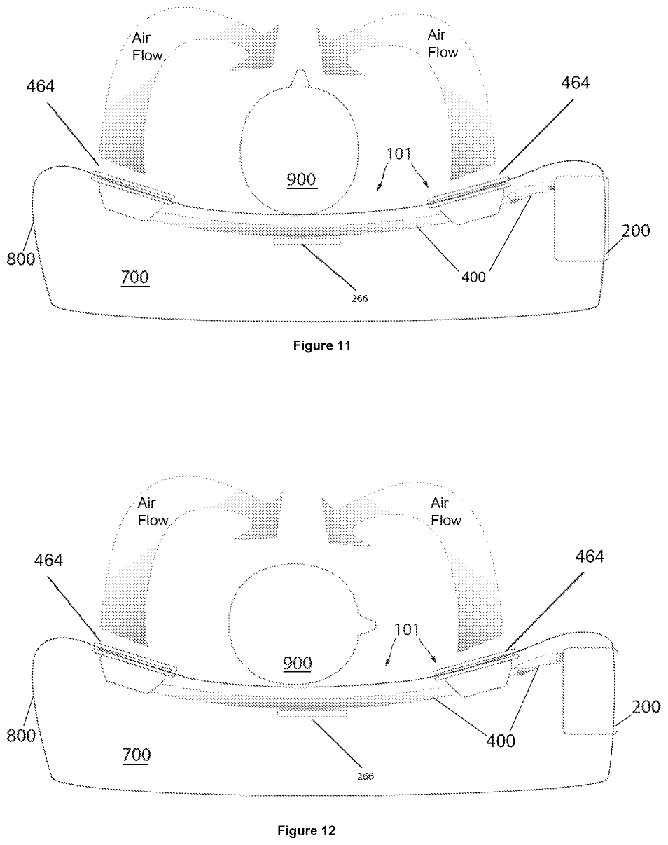

FIG. 12 is another partially transparent front view of the pillow 101 and pillowcase 800 with user's head 900 in contact with the pillow 101 and pillowcase 800. As depicted, in FIG. 12, user's head 900 is rotated to the side. Outlet vent assemblies 464 are rotated inward. Thus, air flow can be focused in a flow pattern surrounding head 900. In general, when supportive frame 700 is displaced or compressed downward from a user's body weight, directional flow can be enhanced towards the user.

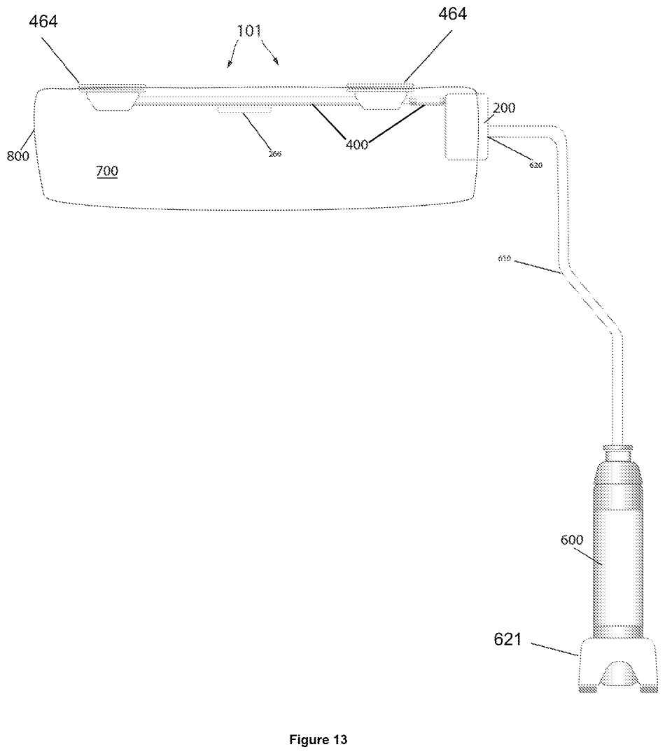

FIG. 13 is partially transparent front view of the pillow 101 and pillowcase 800 connected to oxygen cannister 600. As depicted in FIG. 13, pillow 101 is connected to oxygen cannister 600. Oxygen cannister 600 is supported by optional base 621. Oxygen can be transferred from cannister 600 through tube 610 and connector 620 to gaseous fluidic transfer device assembly 200. Gaseous fluidic transfer device assembly 200 can control the release of oxygen at vents 464 (e.g., in a flow pattern as depicted in FIGS. 11 and 12) when sensor 266 (and/or other sensors) detect a head on pillow 101. In one aspect, a controlled flow can include releasing appropriate dose amounts of oxygen as desired and/or as set by a user (e.g., at the user interface).

In some aspects, various components are (more or fully) external to a pillow, cushion, mattress topper, or other soft item. For example, gaseous fluidic transfer device assembly 200, other filtration unit, power supply, and remote controls can be external to a pillow, cushion, mattress topper, or other soft item. Components of manifold flow path 400 can also extend outside the pillow, cushion, mattress topper, or other soft item. The components of manifold flow path 400 can carry treated air vents at the pillow, cushion, mattress topper, or other soft item.

When components are external to the pillow, cushion, mattress topper, or other soft item, a wired or wireless interface can transfer data between the pillow, cushion, mattress topper, or other soft item and the external components.

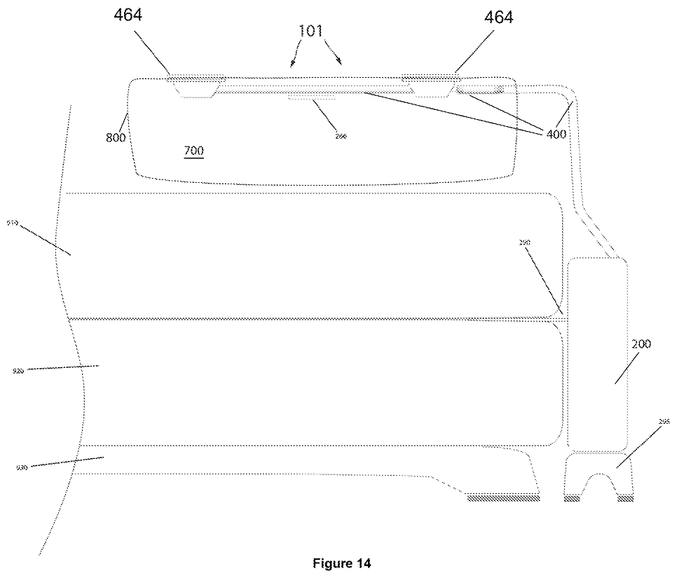

FIG. 14 is partially transparent front view of the pillow 101 and pillowcase 800 connected to an oxygen source and relative to bed components. As depicted in FIG. 14, gaseous fluidic transfer device assembly 200 is fully external to pillow 101 and support frame 700. Gaseous fluidic transfer device assembly 200 is depicted relative to bed components 910, 920, and 930, such as, for example, a mattress, box spring, and bed frame. Portions of manifold flow path 400 are also external to pillow 101 and support frame 700. Gaseous fluidic transfer device assembly 200 is supported by mattress bracket 290 and floor base 295.

Since gaseous fluidic transfer device assembly 200 is external, gaseous fluidic transfer device assembly 200 can be larger in size and can transform, pressurize, and move larger amounts of air. The larger size of the device assembly 200 allows for a larger fan, larger transformative filters, and additional room for supplemental element storage to reside within the unit. In this external configuration, gaseous fluidic transfer device assembly 200 is capable of delivering larger amounts or higher volumes of transformed gaseous elements (e.g., aromas, oxygen, etc.) in fluidic communication with the user. Additional supplemental flow deflectors can be added above the shown assembly to additionally control the vector flow of the air leaving the device vents.

Accordingly, in general, a flexible frame supports a flow manifold which can be used in unison with adjoined or adjacently integrated supportive members utilized to create soft objects of various designs and materials. These types of objects can be in the form of a pillow, mattress topper, even a child's stuff animal and other various stationary and portable soft sided padded embodiments as disclosed in this application. These various designs and embodiments house an integrated flow delivery system which is delivers concentrated, pressurized, and or altered gaseous flowing elements in a controlled and fluidic manner as to help address certain types of sleeping and breathing issues during their resting and sleep states.

More specifically, a mattress topper, stuffed animal, or other padded objects can also include a combination of components described with respect to pillow 101. The mattress topper, stuffed animal, or other padded objects can facilitate delivery of a flow of concentrated, pressurized, and or altered gaseous flowing elements to a user.

The specification and drawings are to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. An air delivery system, comprising:
a vent formed into an external surface of a cushioned support structure;
an air flow tube at least partially contained internally within in the cushion support structure; and
an air transfer device; and
wherein one end of the air flow tube is connected to the vent and another end of the air flow tube is connected to the air transfer device.

2. The air delivery system of claim 1, wherein the air transfer device is fully external to the cushioned support structure.

3. The air delivery system of claim 1, wherein the air transfer device is at least partially contained internally within the cushioned support structure.

4. The air delivery system of claim 1, further comprising a pivotal connector, the pivotal connector connected to the vent and configured to pivot the orientation of the vent.

5. The air delivery system of claim 1, further comprising a sensor embedded in the cushioned support structure, the sensor configured to detect when an external object is in contact with the cushioned support structure and activate the air transfer device.

6. The air delivery system of claim 1, further comprising an oxygen source, and wherein the air transfer device is connected to the oxygen source.

7. The air delivery system of claim 1, further comprising an aroma infuser, and wherein the air transfer device is connected to the aroma infuser.
